(19) United States
(12) Patent Application Publication
(10) Pub. No.: US 2023/0352193 A1
(43) Pub. Date: Nov. 2, 2023

(54) Computational Drug Target Selection

(71) Applicant: EXSCIENTIA AI LIMITED, Dundee (GB)

(72) Inventors: Daniel James CROWTHER, Oxford (GB); David Narganes-Carlon, Oxford (GB); Guillermo Serrano-Na-Jera, Dundee (GB)

(21) Appl. No.: 18/138,705

(22) Filed: Apr. 24, 2023

Related U.S. Application Data
(63) Continuation of application No. PCT/GB2021/052813, filed on Oct. 29, 2021.

(30) Foreign Application Priority Data
Oct. 29, 2020 (GB) GB2017177.3

(51) Int. Cl. G16H 70/40

(52) U.S. Cl. CPC G16H 70/40

(57) ABSTRACT

A method for computational drug target selection includes ingesting publication data from at least one publication data source, the publication data relating to a plurality of publication documents including historical publication documents and current publication documents. The method includes searching the publication data to provide an indication, for each of the publication documents, as to whether the respective publication document is associated with one or more drug targets. The method includes determining an expected publication parameter for each of the one or more drug targets based on the searched publication data from the historical publication documents, and determining an actual publication parameter for each of the one or more drug targets based on the searched publication data from the current publication documents. The method includes evaluating each of the one or more drug targets for selection based on its actual publication parameter relative to its expected publication parameter.

### RELATED APPLICATIONS

This application is a continuation of PCT Patent Application No. PCT/GB2021/052813, filed on Oct. 29, 2021, entitled “Computational Drug Target Selection,” which claims priority to U.K. Patent Application No. GB2017177.3, filed on Oct. 29, 2020, entitled “Computational Drug Target Selection,” each of which is incorporated by reference herein in its entirety.

### FIELD OF THE INVENTION

The invention relates to methods and systems for the computational selection of target molecules or genes, e.g., drug targets, with which molecules, e.g., drugs, are to be designed to interact in an optimal manner.

### BACKGROUND

Drug discovery is the process of identifying candidate compounds for progression to the next stage of drug development, e.g., pre-clinical trials. Such candidate compounds are required to satisfy certain criteria for further development. Modern drug discovery involves the identification and optimization of initial screening ‘hit’ compounds. In particular, such compounds need to be optimized relative to required criteria, which can include the optimization of a number of different properties. The properties to be optimized can include, for instance: activity against a desired biological target; selectivity against non-desired biological targets; low probability of toxicity; and good drug metabolism and pharmacokinetic properties (ADME). Only compounds satisfying the specified requirements become candidate compounds that can continue to the drug development process.

The identification and selection of biological or drug targets against which hit compounds are then to be optimized is therefore a critical step in the drug discovery process; indeed, target identification and prioritization are the first key steps in the drug discovery process and in the development of new pharmaceutical agents. A drug target is something—typically a protein or nucleic acid, for instance—that exists in a living organism to which a drug interacts, e.g., binds. Such interaction with a drug causes a change in behavior of the drug target. A promising drug target may be one that has an association with a particular disease under consideration, e.g., the drug target modifies the disease or plays a role in the pathophysiology of the disease.

The process of selecting a drug target is complicated by the vast number of potential drug targets that are available. For instance, for a human disease there are tens of thousands of genes expressing proteins that could conceivably be the targets for a new drug. Furthermore, as there are many thousands of human diseases classified by medicine then there are many millions—in particular, hundreds of millions—of possible target-disease combinations. The search space of solutions is therefore so large that it is unfeasible to experimentally test each combination or hypothesis.

Conventionally, drug targets have been identified on a case-by-case basis by medicinal chemists interpreting the published scientific literature, e.g., academic journals, and public databases. That is, traditionally a significant amount of target identification has been carried out by individual scientists using their expertise to interpret the scientific literature. However, a growing issue with this approach is in the shear wealth of public data, such as academic papers, that is available to be searched. There are tens of millions of published scientific papers in the area of life sciences, hundreds of thousands of genomes, and many hundreds of databases. Indeed, thousands of peer-reviewed articles are published every day without taking into account other sources of data, such as pre-prints and clinical trial reports. Clearly, it is therefore not possible for humans to keep abreast of all of the available sources of data when selecting drug targets. In other words, the increasing publication rates make it difficult to maintain an overview in order to identify promising new or existing drug targets.

Optimizing the identification and selection of a drug target is crucial in optimizing the overall drug discovery process. In particular, an optimal selection of drug target for a particular drug discovery project can increase the probability of identifying a candidate compound in less time, i.e., in fewer design cycles of the project. In turn, this reduces the associated time and/or cost associated with the particular project.

It is against this background to which the present invention is set.

### SUMMARY OF THE INVENTION

The present invention provides an improved method of identifying biological targets for drugs, possibly in association with particular diseases, in order to reduce the overall time and/or cost associated with the drug discovery process, e.g., to increase the efficiency of identifying a candidate compound as part of a particular drug discovery project. In addition, the invention provides methods for drug discovery. In particular, in methods comprising selecting at least one drug target, the methods may comprise undertaking a drug discovery project based on the at least one drug target; and optionally selecting and/or synthesizing and/or testing potential therapeutic compounds against the at least one selected drug target.

According to an aspect of the present invention there is provided a method for computational drug target selection. The method includes ingesting publication data from at least one publication data source and relating to a plurality of publication documents including historical publication documents and current publication documents. The method includes searching the publication data to provide an indication, for each of the publication documents, as to whether the respective publication document is associated with one or more drug targets. The method includes determining an expected publication parameter for each of the one or more drug targets based on the searched publication data from the historical publication documents, and determining an actual publication parameter for each of the one or more drug targets based on the searched publication data from the current publication documents. The method includes evaluating each of the one or more drug targets for selection based on its actual publication parameter relative to its expected publication parameter.

The method may comprise defining, for each drug target, one or more character expressions as referring to the drug target, and wherein searching the publication data comprises searching the publication data for the one or more character expressions for each drug target.

The method may comprise classifying, for each drug target, each of the one or more character expressions to be a safe character expression or an unsafe character expression. The classification may be based on a likelihood that an instance of the character expression in the publication data refers to the drug target.

In some embodiments, if the searched publication data from one of the publication documents includes a safe character expression, then the publication document is determined to be associated with the drug target.

One or more character expressions may be user-defined to be classified as safe character expressions.

One or more character expression unsafe characteristics may be user-defined to indicate that a corresponding character expression is unsafe. Character expressions in the searched publication data that exhibit one or more of the character expression unsafe characteristics may be classified as unsafe character expressions.

The one or more user-defined character expression unsafe characteristics may include one or more of: a character expression corresponding to a word in a particular natural language; a character expression having fewer than a prescribed number of characters, optionally wherein the prescribed number is three; and a character expression that is defined to refer to at least two different drug targets.

One or more character expression ambiguity characteristics may be defined to ascribe an ambiguity score to one or more of the character expressions. Each of the character expressions may be classified to be a safe character expression or an unsafe character expression based on the correspondingly ascribed ambiguity score.

One or more of the character expression ambiguity characteristics may be user-defined.

The character expression may be classified as an unsafe character expression if its ambiguity score is greater than a prescribed threshold ambiguity score.

The one or more character expression ambiguity characteristics may include one or more of, for each drug target: a total number of publication documents in the publication data that include the defined one or more character expressions referring to the drug target; a number of publication documents in the publication data that includes one of the defined character expressions referring to the drug target, relative to the total number of publication documents in the publication data that include the defined one or more character expressions referring to the drug target; a number of characters in one of the defined character expressions referring to the drug target; a frequency with which each character in one of the defined character expressions referring to the drug target occurs in the publication data, optionally a sum of the frequency for each of the characters in the one character expression, optionally a logarithm of the sum; a number of the defined character expressions for the one or more drug targets that include the one defined character expression; a probability that a publication document in the publication data that includes one of the defined character expressions, other than a selected character expression that is a safe character expression from the defined character expressions referring to the drug target, also includes the selected character expression; and, a probability that a publication document in the publication data that includes the selected character expression also includes the one of the defined character expressions other than the selected character expression.

The method may comprise applying a machine learning algorithm to ascribe the ambiguity score to each of the one or more character expressions based on the one or more character expression ambiguity characteristics.

The machine learning algorithm may use the one or more character expression unsafe characteristics to ascribe the ambiguity score to each of the one or more of the character expressions.

The machine learning algorithm may comprise a positive-unlabeled learning technique.

The machine learning algorithm may comprise application of a random forest classifier.

In some embodiments, after each iteration of the machine learning algorithm, a subset of the ascribed ambiguity scores are inspected by a user to determine whether to manually change any of the subset of ascribed ambiguity scores.

The subset may correspond to a prescribed number of the character expressions having the highest ascribed ambiguity scores.

The publication data for at least some of the publication documents may include citation data indicative of citations made by one publication document to one or more other publication documents from the plurality of publication documents. Searching the publication data may comprise identifying, using the citation data, pairs of publication documents that have been cited by the same publication document.

The method may comprise determining, for each identified pair of publication documents, a co-citation value representative of a number of publication documents that cite both of the pair of publication documents.

The method may comprise assigning pairs of publication documents to one of a plurality of communities of publication documents based on their determined co-citation value and on the publication documents that cite the pairs of publication documents.

In some embodiments, assigning pairs of publication documents to one of the plurality of communities includes application of a greedy optimization algorithm.

The method may comprise determining, for each of the plurality of communities of publication documents, whether to associate the community with one of the drug targets.

The determination may comprise determining which of the defined character expressions referring to the one drug target are present in the publication data of each of the publication documents in the community.

The determination may comprise determining a proportion of the publication documents in the community that include at least one safe character expression in their publication data. In some embodiments, it is determined to associate the community with the one of the drug targets if the proportion is greater than a prescribed threshold proportion.

In some embodiments, searching for the pairs of publication documents includes searching for pairs of publication documents that each include at least one of the character expressions defined as referring to one of the drug targets.

In some embodiments, the publication data for at least some of the publication documents does not include citation data. For each of the publication documents, the method may comprise determining whether to assign the publication document to one of the communities associated with one of the drug targets based on its publication data, in particular on one or more of the defined character expressions referring to the drug target in its publication data.

In some embodiments, if the publication data of the publication document includes at least one instance of a safe character expression, then it is determined to assign the publication document to the one of the communities associated with the one of the drug targets.

In some embodiments, if the publication data of the publication document does not include at least one instance of a safe character expression, then the determination whether to assign the publication document to one of the communities associated with one of the drug targets is performed using a machine learning algorithm.

The machine learning algorithm may comprise a positive-unlabeled learning technique.

The machine learning algorithm may comprise application of a machine learning classifier, optionally at least one of: a logistic regression classifier; an extra tree classifier; a gaussian process classifier; a k-nearest neighbor classifier; a ridge classifier; a random forest classifier; and a support vector machine classifier.

For each drug target, the expected publication parameter may be an expected number of publication documents associated with the drug target, and the actual publication parameter may be an actual number of publication documents associated with the drug target.

For each drug target, the expected publication parameter may be one of: an expected number of clinical trials associated with the drug target; an expected number of review publication documents associated with the drug target; and, an expected number of publication documents linked to a defined size of company; and, the actual publication parameter may be one of: an actual number of clinical trials associated with the drug target; an actual number of review publication documents associated with the drug target; and, an actual number of publication documents linked to the defined size of company, respectively.

In some embodiments, determining the expected publication parameter comprises using a machine learning algorithm trained using the searched publication data from the historical publication documents.

The machine learning algorithm may be a recurrent neural network algorithm.

In some embodiments, evaluating the drug targets for selection comprises ranking the drug targets based on a comparison of their respective actual and expected publications parameters.

The drug targets may be ranked according to a parameter indicative of a difference between their respective actual and expected publications parameters.

The method may comprise determining a target-target co-occurrence parameter between pairs of the drug targets, the target-target co-occurrence parameter being determined based on the indication from the searched publication data which publication documents both drug targets in a pair are associated with. Each target-target co-occurrence parameter may be indicative of the number of publication documents in which both of the drug targets in a pair appear. The method may comprise evaluating the one or more drug targets for selection based on the determined target-target co-occurrence parameters.

The method may comprise searching the publication data to provide an indication, for each of the publication documents, as to whether the respective publication document is associated with one or more diseases.

The method may comprise defining, for each disease, one or more character expressions as referring to the disease. Searching the publication data may comprise searching the publication data for the one or more character expressions for each disease.

The method may comprise determining a target-disease co-occurrence parameter between each of the drug targets and each of the diseases. The target-disease co-occurrence parameter may be determined based on the indication from the searched publication data which publication documents each drug target and each disease are associated with. Each target-disease co-occurrence parameter may be indicative of the number of publication documents in which one of the drug targets and one of the diseases appear. The method may comprise evaluating the one or more drug targets for selection based on the determined target-disease co-occurrence parameters.

The method may comprise applying a topic modeling algorithm to the publication data for the publication documents associated with each of the drug targets to obtain one or more topics associated with each drug target. The method may comprise evaluating the one or more drug targets for selection based on the obtained one or more topics.

The method may comprise determining, for each drug target, errors in association of one or more publication documents with the drug target based on the obtained one or more topics.

The topic modeling algorithm may include at least one of: a latent Dirichlet allocation algorithm; and a non-negative matrix factorization algorithm.

The publication data relating to one or more of the publication documents may include one or more of: a title of the publication document; an abstract of the publication document; and one or more keywords associated with the publication document.

The publication data may include a publication date for each of the plurality of publication documents.

The publication date may define whether each of the publication documents is a historical publication document or a current publication document.

In some embodiments, publication documents having a publication date prior to a predefined threshold date are defined to be historical publication documents.

In some embodiments, publication documents having a publication date after the predefined threshold date are defined to be current publication documents.

In some embodiments, publication documents having a publication date in a predefined threshold date range are defined to be current publication documents.

The at least one publication data source may include at least one online publication data source.

The one or more drug targets may include one or more genes, optionally one or more human genes, optionally one or more proteins encoded by such genes.

The method may comprise using the evaluation of the one or more drug targets to inform selection of at least one of the drug targets for use in a drug discovery project.

The method may comprise designing the drug discovery project by selecting at least one of the drug targets for use in the drug discovery project based on the evaluation.

The method may comprise undertaking the drug discovery project using the at least one selected drug target.

In some embodiments, undertaking the drug discovery project includes selecting, optionally synthesizing, and testing (in silico, in vitro and/or in vivo) compounds against the at least one selected drug target.

According to another aspect of the present invention there is provided a method for identifying a drug/compound having binding affinity for a drug target/target molecule, the method comprising undertaking a drug discovery project (e.g. based on a method for identifying a drug target according to aspects and embodiments disclosed herein) and optionally selecting and/or synthesizing and/or testing compounds against the at least one selected drug target to identify a compound having therapeutic activity against the drug target; wherein ‘therapeutic activity’ may include, without limitation, a desirable binding characteristic (e.g. affinity, selectivity); inhibition characteristic; agonist or antagonist characteristics.

It will be appreciated that any of the features of any aspect or embodiment disclosed herein may be combined with any of the features of any other aspect or embodiment disclosed herein, and all such combinations of features are envisaged and hereby disclosed unless such combination is clearly incompatible.

According to another aspect of the present invention there is provided a non-transitory, computer-readable storage medium storing instructions thereon that when executed by a computer processor causes the computer processor to perform the method described above.

According to another aspect of the present invention there is provided a computer device for drug target selection. The computer device is configured to ingest, receive, or download publication data from at least one publication data source and relating to a plurality of publication documents including historical publication documents and current publication documents. The computer device is configured to search the publication data to provide an indication, for each of the publication documents, as to whether the respective publication document is associated with one or more drug targets. The computer device is configured to determine an expected publication parameter for each of the one or more drug targets based on the searched publication data from the historical publication documents and determine an actual publication parameter for each of the one or more drug targets based on the searched publication data from the current publication documents. The computer device is configured to evaluate each of the one or more drug targets for selection based on its actual publication parameter relative to its expected publication parameter.

### BRIEF DESCRIPTION OF THE DRAWINGS

Examples of the invention will now be described with reference to the following drawings, in which:

FIG. 1 summarizes the steps of a computational drug selection method in accordance with some embodiments.

FIGS. 2(a)-2(f) illustrate a graph database showing relationships between publication documents determined to be associated with a particular gene of interest using the method of FIG. 1.

FIGS. 3(a)-3(d) illustrate a comparison of predicted versus real publication dynamics associated with different genes, determined using the method of FIG. 1.

FIGS. 4(a) and 4(b) illustrate predicted relative to real publication dynamics associated for different genes associated with different groups of diseases determined using the method of FIG. 1.

FIG. 5 illustrates a co-occurrence network showing gene-gene connections and gene-disease connections determined using the method of FIG. 1.

FIG. 6(a)-6(c) illustrate timelines for the number of publications mentioning different genes of interest for different groups of publications each related to different extracted topics as determined using the method of FIG. 1.

### DETAILED DESCRIPTION

Molecular or drug design can be considered a multi-dimensional optimization problem that uses the hypothesis generation and experimentation cycle to advance knowledge. Each compound design can be considered a hypothesis which is falsified in experimentation. The experimental results are represented as structure-activity relationships, which construct a landscape of hypotheses as to which chemical structure is likely to contain the desired characteristics. The process of drug design is also an optimization problem as each project needs a defined product profile—i.e., drug target function—of desired, specified attributes against which hit compounds are analyzed.

The drug discovery process is typically performed in iterations known as design cycles. At each iteration a set of molecules or compounds is synthesized, and their biological properties are measured. The activities are analyzed, and a new set of compounds is proposed, based on what has been learned from previous iterations. This process is repeated until a clinical candidate is found. As well as activity, the measured biological properties can include one or more of selectivity, toxicity, absorption, distribution, metabolism, and excretion.

The drug discovery process is generally time consuming and expensive. Efficiencies that can be found at any stage of the process can therefore help in reducing the time and cost associated with a drug discovery project. Pharmaceutical companies are actively looking for ways to reduce their attrition rates, the time taken for drug development, and the associated development costs.

The selection of drug targets for developing a new drug is the first decision—and arguably the most important single decision—in the drug discovery process. Historically, target identification has been broadly carried out on a case-by-case basis, based on the scientific interpretation of the available literature. However, thousands of peer-reviewed articles are published every day in addition to the publication of pre-prints, patent document data, and clinical trial reports. The online resource PubMed® allows search of, and access to, publication documents in the field of life sciences and biomedical information. PubMed® alone contains access to tens of millions of publication documents—in particular, more than 30 million publications—and the scientific output doubles every nine years or so. This creates a corpus of ‘undiscovered public knowledge’ as it is clearly not feasible for a human, e.g., a medicinal chemist, to keep pace with all of the developments in the published literature. In turn, this makes it more difficult for a human to make an informed decision on the identification and selection of drug targets based on the available literature.

The vast search space of possible drug targets also makes it difficult for a human to make optimal selections. For a human disease, with tens of thousands of genes that could in theory play a role in the behavior of a particular disease, there are many millions of gene-disease combinations that could be investigated but which in practice is clearly not feasible.

The above issues mean that the use of computational methods to analyze the vast amount of available information and the huge number of possible gene-disease combinations becomes an attractive proposition. In particular, there is a high demand for machine learning (ML), artificial intelligence (AI), and other computational methods to exploit the current knowledge and facilitate the maintenance of an overview of this overwhelming volume of literature with a view to optimizing identification and selection of drug targets.

The present invention recognizes that computational methods can be utilized to identify trends in the published literature in respect of potential drug targets, e.g., genes, which can be used to inform selection of drug targets for particular drug discovery projects, for instance. The invention is advantageous in that it provides a computational method for drug target selection that can detect changing trends in relation to certain genes, for instance, which is interpreted to indicate a change in the fundamental assumptions about a particular gene, e.g., a scientific breakthrough regarding a particular gene.

In accordance with the present invention, a first step of the computational drug target selection method comprises ingesting publication data from at least one publication data source. For instance, the publication data source may be an online publication data source, and may include a database such as PubMed® that has access to millions of publication documents in the form of academic or journal articles in the particular field of interest. Publication data may additionally or alternatively be ingested from other sources such as published clinical trial report data, published patent document data and/or published pre-prints of articles. It will be understood that publication data may be obtained and ingested from any suitable source of such publication data, and from any number of these suitable sources.

The information included in the ingested publication data for a given publication document may depend on the particular type of document or the particular source from which the publication data is obtained. For instance, the publication data may be restricted to the data available as open source data for a given publication document, with further information only being accessible behind a paywall.

The ingested publication data relates to a plurality of different publication documents, i.e., each publication document has publication data associated with it. In order to detect trends over time in the literature, the publication documents for which publication data is ingested may be regarded as being split into historical publication documents and current publication documents. Such a split can be useful in making comparisons between trends that may be expected to be observed based on the historical literature versus trends that are actually being observed based on current publications, as will be discussed in greater detail below. It will be understood that the ingestion of historical and current publication data may be performed separately or at the same time.

The publication data associated with at least some of the plurality of publication documents may include a publication date of, or associated with, those publication documents. This may be used to determine or define which documents are identified as historical publication documents and which are identified as current publication documents. The publication date could be a specific day, month, or year of publication of the relevant document, for instance. Purely by way of example, publication documents having a publication date prior to a predefined threshold date may be defined to be historical publication documents, whereas publication documents having a publication date after the predefined threshold date may be defined to be current publication documents. However, the publication date of documents may be used in any suitable way to define whether they are historical or current documents, e.g., publication documents having a publication date in a predefined threshold date range may be defined to be current publication documents.

A next step of the present invention includes searching the ingested publication data to provide an indication, for each of the publication documents, as to whether the respective publication document is associated with one or more drug targets, e.g., genes. That is, the information included in the publication data of each publication document is searched to identify potential associations or links that each publication document has to one or more drug targets. Such a search may be performed in any suitable manner. One option is to search for mentions of the drug targets of interest in the publication data. In this regard, the publication data may include data such as one or more of a title, an abstract, and one or more keywords associated with the publication document. Such information is generally readily available as open source information from online publication databases storing journal articles, for instance, and as such this information may be readily ingested as part of the publication data associated with different publication documents.

In one example, the names of one or more drug targets of interest, e.g., genes of interest, are defined (e.g., by a user) and the content of the publication data is automatically searched for the defined drug target names. For instance, if the defined name of one of the drug targets is found in the publication data associated with a particular publication document, then that drug target may be regarded as being associated with, or linked to, the particular publication document. The defined name for a drug target may for example be an approved symbol, e.g., an approved gene symbol, according to an accepted nomenclature. In the following, ‘gene symbol’ is used to refer to the approved symbol for a particular gene from any of the 19084 human, protein-coding genes accepted by the HUGO Gene Nomenclature Committee; however, it will be understood that this is purely for illustrative purposes and is non-limiting.

A significant obstacle for the automatic analysis of the biomedical literature by computational methods is in the use of non-redundant alternative gene (drug target) synonyms, symbols, and acronyms from different competing sources that can have other meanings in other areas of research. That is, in the literature a single drug target, such as a gene, can be referred to in a number of different ways that are accepted in the field. It can also be the case that one or more of the synonyms for a particular drug target coincides with, or is part of, the term or expression for an entirely different concept, or have an entirely different meaning, in a different context. These factors make it difficult to unambiguously determine via automatic (computational) analysis which publication documents that include references coinciding with a name for a drug target do in fact refer to that drug target.

In order to identify references in the publication data to drug targets of interest that are referred to in a number of different ways, by a number of different names, or in different languages, the method may include defining, for each drug target, one or more character expressions or synonyms as referring to that drug target. These character expressions may be defined by a user and may include any suitable characters that can be searched computationally. For instance, suitable characters may include letters used in one or more natural languages, or other types of symbols. Then, for each drug target, searching the publication data may include searching the publication data for the one or more defined character expressions or synonyms for each drug target.

In this context, where the drug target is a gene, a ‘gene synonym’ may refer to any of the possible gene name variations by which the scientific community refers to, or has referred to, a given gene. Approved gene symbols—as defined above—are also included in the gene synonyms. As an illustrative example, ‘EGFR’ is the approved gene symbol whereas ‘EGFR’, ‘Epidermal Growth Factor Receptor’, ‘ERBB1’, ‘ErbB-1’, ‘c-erbB1’, ‘HER1’, and ‘ERBB’ may be the gene synonyms.

The different character expressions defined as potentially referring to a particular drug target may be obtained from different sources. For instance, in a case in which human genes are of interest, the various character expressions, i.e., synonyms, for different human genes may be gathered from different sources to sample the potential publication documents mentioning human gene names.

As mentioned above, another problem with the automatic analysis of biomedical literature is that instances of accepted synonyms for a drug target in the publication data, i.e., instances of the defined character expressions, may or may not actually refer to the drug target of interest in a particular document. Indeed, it is not uncommon for a particular character expression to be used to refer to two different genes, for instance, in different contexts. Therefore, it is imperative to disambiguate biomedical entities in the scientific literature in order to accurately analyze publication dynamics of different drug targets.

Each defined character expression for a drug target, e.g., each different synonym for a gene, may be regarded as having a different level of ambiguity associated with it. That is, some synonyms that are found in the literature that have meanings outside of a particular gene of interest, for instance, can be regarded as having a greater level of ambiguity associated with them as there is a greater likelihood that instances of those synonyms in the literature are referring to something different from the gene of interest. On the other hand, when a particular character expression or synonym does not have a (common) meaning outside of referring to a particular gene of interest, then such a synonym may be regarded as having a low level of ambiguity in that instances of that synonym in the literature are therefore likely to be referring to the gene of interest.

A level of ambiguity associated with a gene synonym (drug target character expression) may arise for different reasons. For instance, a so-called ‘promiscuous gene name (homonym)’ may be regarded as any gene name that is a synonym of more than one gene. This could include previous official gene symbols (according to an accepted nomenclature) as these will not have been expunged from the literature. As an illustrative example, ‘CDH3’ and ‘cadherin3’ are promiscuous for gene symbols ‘CHD15’ and ‘CHD3’. Also, ‘ARP1’ is a gene synonym for the gene symbols ‘NR2F2’, ‘ACTR1A’, ‘ACTR1B’, ‘ANGPTL1’, ‘APOBEC2’, ‘ARFRP1’, and ‘PITX2’. As another example, a so-called ‘nested gene synonym’ may be regarded as a gene synonym that is part of another gene synonym. For instance, ‘insulin’ is a nested gene synonym of ‘insulin receptor’. Also, ‘TNF’ is a nested gene synonym of ‘TNF Receptor Superfamily Member 1A’ (gene symbol ‘TNFRSF1A’) and ‘TNF Receptor Associated Factor 2’ (gene symbol ‘TRAF2’).

In order to allow for a more accurate analysis of the searched publication data, the method of the invention may therefore include classifying, for each drug target (e.g., gene), each of the one or more defined character expressions (e.g., gene synonyms) found in the publication data to be a safe character expression or an unsafe character expression. The classification is based on a likelihood that an instance of the character expression in the publication data refers to the drug target, i.e., a level of ambiguity associated with the character expression. In particular, a safe character expression may have a relatively low level of ambiguity associated with it, whereas an unsafe character expression may have a relatively high level of ambiguity associated with it. For instance, where the character expressions are gene synonyms, an ‘unsafe gene synonym’ may include a gene synonym that has a different meaning in other areas of research or in a different context, e.g., a word that appears in the English dictionary. As an example, the ‘STAR’ gene symbol may be regarded as being an unsafe character expression as opposed to its gene synonym ‘Steroidogenic Acute Regulatory Protein’. As another example, ‘CCP4’ may be regarded as being unsafe as it is both a gene synonym and the name for crystallography software.

If the searched publication data from one of the publication documents is determined to include a safe character expression (for a particular drug target), then that publication document may be determined to be associated with that drug target. In other words, that publication document is regarded as being linked to, or related to, the particular drug target under consideration, e.g., gene of interest. For a particular drug target, at least some of the defined character expressions that potentially refer to the drug target, i.e., in that they appear in the literature, may be regarded as definitely being safe character expressions. In particular, one or more of the character expressions may be user-defined to be classified as safe character expressions. That is, there are certain character expressions that have no ambiguity—or a very low level of ambiguity—such that it is known a priori that instances of those character expressions in the publication data do in fact refer to the relevant drug target regardless of the context in which those instances appear in the publication data. Such character expressions can therefore automatically be classified as safe character expressions when they are found in the publication data. This means that, when such a character expression is found in the publication data of a certain publication document, it may be automatically determined that the publication document is associated with, or linked to, the drug target for which the character expression is a defined synonym.

One or more characteristics of character expressions may be defined, e.g., by a user, to be indicative that character expressions exhibiting such characteristics have a high level of ambiguity such that they are unsafe. In particular, character expressions in the searched publication data that exhibit such ‘character expression unsafe characteristics’ may be classified automatically as unsafe character expressions. An example of a user-defined character expression unsafe characteristic may be any character expression corresponding to a word in a particular natural language, e.g., a word in the English dictionary (see the ‘STAR’ example above). Another example of a user-defined character expression unsafe characteristic may be a character expression having fewer than a prescribed number of characters. For instance, the prescribed number may be three, or any other suitably-defined number. A further example of a user-defined character expression unsafe characteristic may be a character expression that is defined to refer to at least two different drug targets (see ‘promiscuous gene name’ mentioned above). In this way, character expressions including at least one of the defined unsafe characteristics are regarded as being definitely unsafe. It will be understood that any suitable characteristics of a character expression may be defined as being indicative that the character expression is highly ambiguous, and so unsafe.

There will likely be a significant number of the defined character expressions that are not regarded as definitely safe or definitely unsafe according to the definitions above. In this regard, a level of ambiguity associated with the remaining character expressions may be determined or calculated in order to classify these character expressions as being safe or unsafe. One option to determine which character expressions or synonyms have a potentially high level of ambiguity—such that they are regarded as being unsafe—is to perform feature engineering to obtain variables that characterize unsafe synonyms and then to ascribe a level of ambiguity to each of the synonyms based on the obtained variables. For instance, longer gene names may be less likely to be ambiguous. More generally, character expression ambiguity characteristics may be defined in any suitable manner to ascribe an ambiguity score to one or more of the character expressions. This may be by user definition, e.g., based on the feature engineering, or otherwise. Each of these character expressions may then be classified to be a safe character expression or an unsafe character expression based on the correspondingly ascribed ambiguity score. For instance, a character expression may be classified as an unsafe character expression if its ambiguity score is greater than a prescribed threshold ambiguity score.

A machine learning algorithm may be applied to ascribe the ambiguity score to each of the character expressions or synonyms based on the obtained character expression ambiguity characteristics, e.g., by feature engineering. In particular, the machine learning algorithm may use the character expression unsafe characteristics to ascribe the ambiguity score to each of the character expressions not yet classified as safe or unsafe. That is, an ambiguity score is ascribed to synonyms in an unlabeled set of synonyms, i.e., those not in a set of synonyms previously labelled as safe or a set of synonyms previously labelled as unsafe. The ambiguity score is then used to label the as yet unlabeled synonyms as safe or unsafe. To achieve this, the machine learning algorithm may include application of a positive-unlabeled learning technique, e.g., positive-unlabeled bagging strategy, and a classification scheme, e.g., a random forest classifier.

The machine learning algorithm may be run as an iterative process. After each iteration of the algorithm, a subset of the ascribed ambiguity scores may be inspected by a user to determine whether to manually change any of them, i.e., to correct classifications made by the algorithm in order to train the algorithm and increase its accuracy for subsequent iterations. For instance, the subset may correspond to a prescribed number of the synonyms or character expressions having the highest ascribed ambiguity scores (and therefore considered by the algorithm to be the most unsafe).

The character expression ambiguity characteristics—obtained by feature engineering, for instance—may include a total number of publication documents in the publication data that include a defined expression referring to a specific drug target. The ambiguity characteristics may include a number of publication documents in the publication data that includes one of the defined character expressions referring to the drug target, relative to the total number of publication documents in the publication data that include the defined character expressions referring to a specific drug target. The ambiguity characteristics may include a number of characters in one of the defined character expressions referring to a drug target. For instance, shorter expressions may in general be regarded as more ambiguous than longer ones. The ambiguity characteristics may include a frequency with which each character in one of the defined character expressions referring to the drug target occurs in the publication data. More specifically, a sum of the frequency for each of the characters in a particular character expression may be considered, i.e., a frequency score for the entire expression. Any suitable metric using this overall frequency score may be used, e.g., a logarithm of this overall score. For instance, synonyms or character expressions including less common characters may be less ambiguous than those synonyms composed entirely of characters commonly found in the publication data (or more generally considered as being common). A further ambiguity characteristic for a particular character expression or synonym may be based on a number of the defined character expressions for the drug targets that include the particular defined character expression. In other words, an ambiguity characteristic may be based on the number of nested synonyms (as defined above) relevant to a particular synonym, i.e., the number of other gene synonyms that contain the particular gene synonym under consideration. Another ambiguity characteristic may be a probability that a publication document in the publication data that includes one of the defined character expressions, other than a selected safe character expression, also includes the selected safe character expression. Expressed differently, an ambiguity characteristic may be the conditional probability of finding the gene synonym of interest in the publication data for a specific publication document given that one of the (other) gene synonyms for the same gene symbol (as defined above) appears in the text. A further ambiguity characteristic may be in essence the ‘reverse probability’ of the above, i.e., a probability that a publication document in the publication data that includes the selected character expression (i.e., the synonym under consideration) also includes another one of the defined character expressions for that drug target. Expressed differently, an ambiguity characteristic may be the conditional probability of finding one of the (other) gene synonyms for the same gene symbol in the publication data for a specific publication document given that the gene synonym of interest appears in the text. As a final example, an ambiguity characteristic may be based on whether the character expression under consideration is the accepted character expression for a particular drug target, e.g. whether the gene synonym under consideration is the gene symbol.

The method of the invention may use the labelled character expressions—i.e., labelled safe or unsafe depending on the associated ambiguity of the expression—to unambiguously associate or link each drug target, e.g., human gene, to a subset of the publication documents for which publication data has been ingested. To do this, an approach based on a network of co-citations may be used. That is, the citations of a publication document may be used to more accurately determine whether mentions of character expressions for a particular drug target in the publication data of that publication data actually mean that the particular drug target is linked to the publication document (or whether the character expression is being mentioned in a different context such that it does not actually refer to the drug target). Specifically, the co-citation approach—described in more detail below—may be used to reduce or eliminate ‘false positives’ from the searched publication data, i.e., publication documents whose publication data mentions a defined character expression (gene synonym)—which is indicative that a publication document may be associated with the drug target (gene) relevant to the defined character expression—but which are in fact not associated with or linked to the drug target. This approach may be regarded as being based on an assumption that publication documents including ‘false positives’ will tend to belong to different communities of publications relating to different research fields from publication documents including ‘true positives’, i.e. publication documents including defined character expressions in the text that do in fact refer to a drug target of interest. In this way, an identified community of publication documents may be determined (as a whole) to be linked to, or to not be linked to, a gene of interest.

In order to analyze the particular citations made by the different publication documents, the ingested publication data for at least some of the publication documents may include citation data indicative of citations made by one publication document to one or more other publication documents from the plurality of publication documents. The method may involve identifying so-called ‘co-citations’ in the publication data. A co-citation may be regarded as an occurrence of two publication documents both being cited by a third document. That is, if ‘Publication A’ and ‘Publication B’ are in the list of references of ‘Publication C’, then there is a co-citation between ‘Publication A’ and ‘Publication B’. The step of searching the publication data may therefore include identifying, using the ingested citation data, pairs of (first and second) publication documents that have been cited by the same (third) publication document. In particular, as it is desired to obtain communities of publications each referring to a particular drug target, this step of searching for the pairs of publication documents includes searching for pairs of publication documents that each include at least one of the character expressions (gene synonyms) defined as referring to one of the drug targets (genes).

A co-citation network may be obtained using the identified co-citations, i.e., pairs of publication documents. For each identified pair of publication documents, a co-citation value representative of a number of (different) publication documents that cite both of the pair of publication documents may be determined. That is, a weighted co-citation graph may be obtained where the weight of the edges represents the frequency of two publications being cited simultaneously (co-cited) by a third publication. When two publications are repeatedly co-cited it is assumed that this strongly suggests that both belong to the same field of study. In turn, this means that it is assumed that both publications in the co-cited pair are either ‘true positives’ or ‘false positives’.

Once a co-citation network is achieved, pairs of publication documents are to be assigned into different communities of publication documents. Each community includes publication documents that include instances of defined character expressions for a particular drug target; however, it may be the case that not all of the communities include publication documents that are in fact associated with the particular drug target, i.e., some communities may be composed of documents whose instances of the character expressions are in a context different from the particular drug target.

In this way, the method may therefore include assigning pairs of publication documents to one of a plurality of communities of publication documents based on their determined co-citation value and on the publication documents that cite those pairs of publication documents. This may be performed automatically using an appropriate community detection technique. For instance, assigning pairs of publication documents to one of the communities may include application of a (fast) greedy optimization algorithm.

Once a number of communities of publication documents have been obtained, the identified communities need to be distinguished from one another. In particular, the method may include determining, for each of the plurality of communities of publication documents, whether to associate that community with one of the drug targets. To do this, the relative ‘safety’ of character expressions (determined as described above) that are present in the publication documents of a particular community may be used. This can involve determining or identifying which of the defined character expressions referring to a particular drug target are present in the publication data of each of the publication documents in a particular community. A determination as to how many safe character expressions are in a community may be used to determine whether that community is associated with the relevant drug target. For instance, a proportion of the publication documents in the community under consideration that include at least one safe character expression in their publication data may be determined. It may then be determined to associate that community with the drug target of interest if the determined proportion is greater than a prescribed threshold proportion. Alternatively, one or more of the communities having the highest proportions of safe character expressions may be regarded as being associated with the relevant drug target.

An issue that may arise with the co-citation approach described above is that the publication data of some of the publication documents may not include citation data, i.e., details of citations made by a particular publication document. This may be a particular issue in a case in which publication data of open-access publications is ingested as citation data is often not available from such sources.

Therefore, for each of the publication documents whose publication data does not include citation data, the method may include determining whether to assign the publication document to one of the communities associated with one of the drug targets based on its publication data, in particular on one or more of the defined character expressions referring to that drug target in its publication data. For instance, if the publication data of a publication document includes at least one instance of a safe character expression, then it may be determined to assign the publication document to one of the communities associated with the relevant drug target. On the other hand, if the publication data of a publication document does not include a safe character expression, then the determination whether to assign the publication document to one of the communities associated with the relevant drug target may be performed using a machine learning algorithm, e.g., a positive-unlabeled learning technique. The machine learning algorithm may apply a machine learning classifier, such as one of a logistic regression classifier, an extra tree classifier, a gaussian process classifier, a k-nearest neighbor classifier, a ridge classifier, a random forest classifier, and a support vector machine classifier. That is, a positive-unlabeled bagging approach may be used to train multiple classifiers to associate the disconnected publications (without citation data) with the previously computed co-citation network components using the words/expressions contained in the publication data, e.g., title, abstract, etc.

The above steps to search the ingested publication data provides an accurate indication of which publication documents in the literature are associated with given drug targets, e.g., genes. In turn, this allows for a more accurate and reliable analysis of the publication dynamics over time for one or more drug targets, e.g., the publication rate associated with a given gene over time.

In accordance with the invention, a next step of the method therefore includes determining an expected publication parameter and an actual publication parameter for each drug target of interest based on the searched publication data. In particular, the expected publication parameter is determined based on publication data from the historical publication documents. Specifically, historical publication dynamics for a particular gene, for example, are calculated using the historical publication documents, and then these historical publication dynamics are used to determine or predict the expected publication parameter, e.g., by extrapolation. As an illustrative example, the publication dynamics for a given gene for each of a number of successive years may be calculated using the historical publication data, e.g., using publication dates associated with historical publication documents in the publication data, and these calculated (historical) publication dynamics can be used to predict current publication dynamics for that given gene. Determining the expected publication parameter may be performed using a machine learning algorithm trained using the searched publication data from the historical publication documents, e.g., a recurrent neural network algorithm. The actual publication parameter is determined based on publication data from the current publication documents.

The expected and actual publication parameters can be a measure or indication of any one or more aspects of the publication dynamics associated with a particular drug target. For instance, the expected and actual publication parameters may be an expected and actual number of publication documents, respectively, e.g., the number of publication documents in a given year. Alternatively, or in addition, the expected and actual publication parameters may include an expected and actual number of clinical trials associated with the particular drug target under consideration, an expected and actual number of review publication documents associated with the particular drug target, and an expected and actual number of publication documents linked to a defined size of company. In each case, the relevant information needs to be available in the ingested publication data in order to determine the relevant parameter. For instance, the publication data for some publication documents may indicate whether the publication document is associated with a large- or medium-sized pharmaceutical company. As an illustrative example, if a manuscript with author affiliations to a big pharmaceutical company cites other publications, then these citations may be categorized as ‘big pharmaceutical company’ citations. Conversely, publications citing this manuscript whose authors are affiliated to a big pharmaceutical company may not be categorized as ‘big pharmaceutical company’ citations.

In accordance with the invention, in order to detect incoming trends in the literature the method then includes evaluating each of the drug targets of interest for selection based on its actual publication parameter relative to its expected publication parameter. For instance, evaluating the drug targets for selection may include ranking the drug targets in a (prioritized) list based on a comparison of their respective actual and expected publications parameters. In particular, a drug target may be considered as potentially interesting for selection if there is a (significant) difference between its respective actual and expected publications parameters as this may mean that there has been a step change in interest in the drug target relative to what may be expected according to historical publication data.

In general, it may be found that the described method produces accurate predictions of the publication dynamics, i.e., the actual publication parameter is generally in line with the expected publication parameter. However, for a small subset of drug target, e.g., genes, the actual or real number of publications or citations may be significantly higher than expected. When the actual number of publications or citations exceeds the predictions, this may be interpreted that the publication dynamics have changed substantially in a way that cannot be explained simply by the publication history of a gene of interest, for instance, implying that a meaningful discovery in the field may have recently occurred. The term ‘trendiness’ may be defined as the probability of a fold-change between the predicted and real number of publications and citations for a given gene. This metric can be used to identify the ‘trendiest’ genes in the academic community (using all publications), or in the pharmaceutical industry (using publications coming from pharmaceutical companies).

The method may include using the evaluation of the drug targets to inform selection of at least one of the drug targets for use in a drug discovery project, for instance based on a ranked list of the trendiest genes. In particular, the method could include designing the drug discovery project by selecting at least one of the drug targets for use in the drug discovery project based on the evaluation. The method may include undertaking the drug discovery project using the at least one drug target as selected, at least in part, based on the above evaluation. Such a drug discovery project can include selecting compounds and testing them against the at least one selected drug target, for instance, e.g., to identify a compound potentially having therapeutic activity against a disease target. The methods of this disclosure may then involve synthesizing at least one compound potentially having binding activity against the selected drug target.

It can also be informative to consider the relationship between two different drug targets when analyzing particular drug targets for selection. In particular, it may be found that genes of interest may cluster together in association networks. Hence it can be informative to have an insight into the association that one gene has with other genes in the literature as this could mean that identification of one gene whose publication dynamics has undergone a fold change may lead to one or more further genes whose publication dynamics have changed in a manner that means they are of interest.

In this regard, analysis of the publication dynamics of drug targets may include determining a target-target co-occurrence parameter between pairs of the drug targets. Such a parameter may be determined based on the indication from the searched publication data which publication documents both drug targets in a pair are associated with, i.e., publication documents with which two different drug targets are associated. Each target-target co-occurrence parameter may be indicative of the number of publication documents in which both of a pair of drug targets appear. The evaluation of drug targets for selection may then be based on the determined target-target co-occurrence parameters.

Drug targets of potential interest to the pharmaceutical industry may be drug targets that can be associated with particular diseases. The described method of searching publication data to associate drug targets with publications can also be applicable to associate particular diseases with publications. In this regard, the method may include searching the ingested publication data to provide an indication, for each of the publication documents, as to whether the respective publication document is associated with one or more diseases. In a similar manner to the method for drug targets above, this may involve defining, for each disease, one or more character expressions as referring to the disease, searching the publication data for the character expressions for each disease.

As a non-limiting illustrative example, disease names and their synonyms may be obtained from the Medical Subject Headings (MeSH) ontology at the Bioportal. MeSH ontology contains 4818 different disease nodes at different levels of the ontology. For instance, a dictionary for each disease may be created with the preferred and alternative names. The diseases can then be disambiguated in the publication data (e.g., title, abstract, etc.) using corresponding techniques described above for genes.

The method may then include determining a target-disease co-occurrence parameter between each of the drug targets and each of the diseases. Such a parameter may be determined based on the indication from the searched publication data which publication documents each drug target and each disease are associated with, each target-disease co-occurrence parameter being indicative of the number of publication documents in which one of the drug targets and one of the diseases appear. The evaluation of drug targets for selection may then be based on the determined target-disease co-occurrence parameters.

It may be desired to gain a greater insight as to why the publication of particular drug targets have changed, perhaps in association with a certain disease, in order to evaluate drug targets for selection. In this way, groups of publications that mention a gene of interest may be analyzed. For instance, the method may include applying a topic modelling algorithm to the publication data for the publication documents associated with a drug target of interest to obtain one or more topics associated with the drug target, and the evaluation of the drug targets for selection may then be based on the obtained topics. A topic may be regarded as a collection of similar words, specific to a group of documents. Non-negative matrix factorization may be used to generate a set of latent topics for each query. In particular, the topic modelling algorithm may include a latent Dirichlet allocation algorithm and/or a non-negative matrix factorization algorithm. The topic detection can also be used to determine, for a drug target, errors in association of one or more publication documents with the drug target according to the search publication data based on the obtained one or more topics, and so can further aid the accuracy of drug target association in the literature.

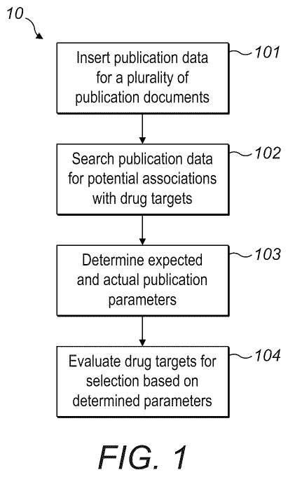

FIG. 1 summarizes the steps of a computational drug target selection method 10 according to the invention. At step 101, publication data is received, ingested or downloaded from at least one publication data source, such as an online database storing publication documents, e.g., articles, journal papers, etc. The publication documents include historical publication documents and current publication documents. The publication data can include publication dates, authorship, titles, abstracts, keywords, citations, etc. in connection with publication documents.

At step 102, the received publication data is searched to provide an indication, for each of the publication documents, as to whether the respective publication document may be associated with one or more potential drug targets, e.g., genes. In particular, this can involve searching the publication data for mentions or instances of one or more defined character expressions for each drug target. A mention of one of these character expressions in the publication data for one of the publication documents indicates that the publication document may be associated with the particular potential drug target. Further steps may be performed to determine whether the publication document is in fact associated with the potential drug target. For instance, a relative ‘safety’ of the character expression in the publication data may be established (as described above) to indicate a confidence that the character expression in the publication data does indeed refer to the potential drug target of interest. Further steps to cluster the publication documents into communities based on the searched publication data may be performed to determine whether clusters of publication documents are in fact associated with, or linked to, the drug target of interest. In general, searching the publication data is used to establish a group of publication documents that are linked to each of one or more potential drug targets.

At step 103, an expected publication parameter for each potential drug target is determined based on the searched publication data from (i.e., connected to) the historical publication documents. Also, an actual or real publication parameter for each potential drug target is determined based on the searched publication data from the current publication documents. The publication parameters may be any suitable parameters for describing publication dynamics (over time) for each potential drug target. For instance, the publication parameters may indicate the number of publication documents each calendar year that are associated with a particular drug target. The expected or predicted publication parameter may be determined by determining the historical publication dynamics for a drug target based on the historical publication data and extrapolating these dynamics to predict the present or future publication dynamics.

At step 104, each potential drug target may be evaluated for selection based on its actual publication parameter relative to its expected publication parameter. In particular, differences between the expected and actual parameters for a given potential drug target may be indicative of a change in assumptions about the drug target and may indicate interest in further investigation for selection as a drug target. The evaluation can involve creating target lists of potential drug targets based on the above analysis (i.e., differences between predictions and actual values, and also perhaps based on the confidence of the predictions) in order to prioritize potential drug targets for selection, for instance in association with any disease or biological mechanism of choice. The evaluation can inform selection of drug targets for various applications, e.g., designing and performing a particular drug discovery project.

The method of the invention may be implemented on any suitable computing device, for instance by one or more functional units or modules implemented on one or more computer processors. Such functional units may be provided by suitable software running on any suitable computing substrate using conventional or customer processors and memory. The one or more functional units may use a common computing substrate (for example, they may run on the same server) or separate substrates, or one or both may themselves be distributed between multiple computing devices. A computer memory may store instructions for performing the method, and the processor(s) may execute the stored instructions to perform the method.

Many modifications may be made to the above-described examples without departing from the scope of the appended claims.

In the following, a specific non-limiting example of the above-outlined computational drug target selection method is described.

### Example 1

PubMed® baseline released in December 2019 contains more than 30 million publications, around 170 million citations from open source data, almost 9 million authors, and almost 300 million MeSH annotations. PubMed® can be converted into a graph database using the graph database platform Neo4J to efficiently query for relationships such as authorship, references, or annotations. The resulting database contained five different node types: publications; authors; human protein-coding genes; human diseases; and Medical SubHeadings (MeSH) terms. The publication nodes have multiple attributes that were extracted from the PubMed® baseline: PubMed ID; title; abstract; keywords; authors; affiliations; the date of publication; the journal; and the article type (e.g., article, review, or clinical trial). An attribute aggregating affiliation data was also included to know whether a pharmaceutical company was involved in the authorship of the publications. There are five types of relationships (edges): cited by (from publication to publication); published (from authors to publications); MeSH annotation (from MESH terms to publications); gene annotation (from genes to publications); and disease annotation (from diseases to publications). In the preparation of this database, a disambiguation pipeline to unequivocally link human protein-coding genes symbols and human diseases to individual publications was implemented.

Human gene synonyms were gathered from different sources (Ensembl, UniProt, HGCN, Entrez and OpenTargets) to sample the potential publications mentioning human gene names. It is noted that human genes have around 10 synonyms each on average, and many of these synonyms are ambiguous (when considered out of context). More than 30% of gene symbols have at least one promiscuous synonym, around 10% of the gene symbols have another meaning in a different context and have at least one gene synonym in the English dictionary, and almost 50% of gene symbols have a nested synonym. Combining these problems, almost 60% of the 19082 gene symbols have at least one of these types of ambiguity. To determine which synonyms are potentially ambiguous, feature engineering is performed to obtain variables that characterize unsafe synonyms (e.g., longer gene names are less likely to be ambiguous). Next, a positive-unlabeled bagging (PU) strategy with a random forest classifier with the engineered features was used to calculate the probability of a gene synonym being ‘unsafe’.

In more detail, 19082 protein-coding human genes annotated by HUGO Gene Nomenclature Committee (HGNC) were used. Gene synonyms which are identical to disease names contained in the Medical Subject Headings (MeSH) database were eliminated. This mainly occurs when genes are named after diseases that they are associated with, e.g., ‘Li Fraumeni syndrome’ as a gene synonym for gene TP53 or ‘Madan syndrome’ in ‘FBN1’.

Gene synonyms were classified into ‘safe’ or ‘unsafe’ categories using a modified version of positive-unlabeled (PU) learning with bootstrap-aggregating. PU learning is a form of semi-supervised learning which iteratively finds positive examples within a-priori unlabeled data. To build a binary classifier able to distinguish the unlabeled class (U) into unsafe (P, positive) and safe (N, Negative) classes, a series of features were engineered, such as the combined frequency of the characters in a gene synonym (example: ‘ZNF’ will be safer than ‘EDA’ because ‘Z’ and ‘F’ characters are less frequent in PubMed® corpus than ‘E’, and ‘A’) or the probability of a gene synonym given that other gene synonym appeared in the text (the probability of ‘STAR’ given ‘Steroidogenic Acute Regulatory Protein’ is high but the probability of ‘Steroidogenic Acute Regulatory Protein’ given ‘STAR’ is low because ‘STAR’ is more ambiguous).

The PU learning was run for five iterations with a random forest classifier. The pure positive class (unsafe) was constructed combining gene synonyms present in the English dictionary, gene synonyms with fewer than three characters, and promiscuous gene synonyms. In an active learning fashion, after each iteration, the top 1000 examples with the highest probability of being unsafe were manually relabeled if they were wrongly classified. For example, true positive unsafe synonyms like gene families (e.g., ‘G protein coupled receptor’), phenotypes (e.g., ‘Williams Beuren Syndrome’) and other biological entities (e.g., ‘Cell surface antigen’) were included in the true positive set for the next iteration. False positives like ‘thymopoietin’ or ‘tubulin alpha-1C chain’ were included into a new true negative class for the remaining iterations.

After the five iterations, a gene synonym was considered unsafe if: (i) it is included in the English dictionary; (ii) it is a word with fewer than three characters; (iii) the predicted score for the random forest classifier was higher than 0.5; and (iv) it is a promiscuous gene synonym.

To link every human gene to a subset of publications a disambiguation pipeline based on co-citation networks and machine learning was implemented. The titles, abstracts and keywords of the publications that had a match for any of the synonyms were gathered using regex with ElasticSearch. In particular, an ElasticSearch API search engine was used to retrieve PubMed® IDs of publications containing a gene or disease synonym in their title, abstract or keywords. These PubMed® IDs were later used to retrieve the publications' attributes from Neo4J using Cypher language through its python driver. Regular expressions were used to avoid nested name ambiguity with lookarounds and fuzzy matching to account for case and punctuation and letter case variations (e.g. ‘ErbB-1’, ‘erbB1’, ‘ERBB1’, ‘ErbB 1’).

To detect communities of publications, co-citation networks were used, i.e., a weighted graph where the weight of the edges represents the frequency of two publications being cited simultaneously (co-cited) by a third publication. The fast greedy modulation algorithm from iGraph was used to determine communities in the co-citation network and distinguished communities of publications focusing on the gene of interest by detecting the presence of ‘safe gene synonyms’ in their titles and abstracts. Each of the publications in a community were labelled with the gene symbol of interest if the ratio of publications mentioning at least one safe synonym with respect to publications that only mention unsafe synonyms was higher than 0.1%.

Finally, because only citations from open-access publications contained in PubMed Central (PMC) were used, 46% of the publications were disconnected in the PubMed® co-citation graph. Disconnected publications that mention a safe-synonym were automatically linked to the gene symbol of interest. The rest of the disconnected publications were linked to the gene of interest using a PU approach bagging strategy with a binary logistic regression classifier based on the words in the text corpus (keywords, titles and abstracts) of communities already linked to the gene of interest and discarded communities. All available machine classifiers in Scikit Learn were used but logistic regression was selected due to its speed to accuracy ratio.

Each corpus was pre-processed by: (i) removal of non-alphanumeric characters; (ii) tokenization or split by whitespace; (iii) deletion of stop words from NLTK (natural language toolkit); (iv) lower case conversion; (v) deletion of tokens whose length is less than three characters; (vi) deletion of tokens representing integers; and (vii) stemming (e.g., ‘disambiguate’, ‘disambiguations’, and ‘disambiguating’ are converted to ‘disambiguat’). List of tokens (uni-, bi-, tri-, tetra-grams) with at least 2 counts and a frequency lower than 0.6 in the complete corpus were vectorized using TF IDF (term frequency-inverse document frequency). When there were fewer than 1000 unlabeled publications in the training set for the gene of interest, an auxiliary negative class was generated to augment the negative examples in the training data. This auxiliary negative class comprised a random sample of 1000 publications that mentioned genes different from the gene of interest.

To test the performance of the disambiguation method the disambiguation results were compared with the gene-publication annotations from GeneRif (manually curated annotations), DISEASES (computational annotations), and UniProt (computational and manually curated annotations). On average, the disambiguation method recovers more than 85% of all publications contained in these databases. Both GeneRif and Uniprot annotation do not necessarily contain a gene-synonym in the title or abstract, therefore those publications are out of the described method. Disambiguation results present on average a 70% precision with UniProt, the only collection of disambiguated publications of a similar magnitude. Finally, the disambiguated gene-publication annotations were included into the graph database.

These vectors were fed to all available machine learning classifiers from the Python library sklearn: Extra Tree Classifier, Gaussian Process Classifier, K-Nearest Neighbor, Logistic Regression, Ridge Classifier, Random Forest Classifier, and Support Vector Machine. All classifiers were trained with hyper-parameter tuning and 3-fold cross-validation to avoid over-fitting in each of the 50 PU-bagging iterations. Loss functions were modified to account for the imbalance of the classes. The logistic regression (LOG) classifier was selected for the disambiguation method given its accuracy-speed balance.

The same procedure used for gene entity recognition was used to detect disease entities, co-citation networks and machine learning. The Medical Subject Headings (MeSH) ontology was downloaded by querying their Rest-API available at BioOntology. Each disease was a node in the ontology. The disease synonyms were obtained from the Concept List Terms' field in the ontology to gather the preferred and the alternative ways of denoting the disease. Further synonyms of the diseases were generated by reversing the order of synonyms with commas: ‘Insipidus, Diabetes’ to ‘Diabetes Insipidus’.

Co-occurrence of genes and diseases was computed using the simultaneous occurrence of gene/disease tags in publications after disambiguation, normalized by the total number of publications presenting those tags. Mutual information metrics for gene-gene and gene-disease associations were also computed.

Every disease MeSH term was associated with its lowest ancestor in the MeSH ontology under the node Disease. After computing the gene-disease co-occurrence, each gene was linked with the most frequent ancestor disease term.

To detect incoming trends in the literature the publication dynamics of a given human gene from the disambiguated graph database were gathered. These time series include the number of publications, clinical trials, reviews, and publications from big and medium-sized pharmaceutical companies, as well as citations of publications coming from the mentioned categories per calendar year.

For most genes, the model produces accurate predictions of the publication dynamics, but for a small subset of genes the real number of publications or citations is significantly higher than expected. The trendiness of a gene can be regarded as the probability of observing the magnitude of fold-change between the predicted and the real number of publications for that given gene. The error in the predictions is inevitably higher with genes associated with small numbers of publications. To correct for this, five bins were generated based on the initial number of publications (percentiles 20, 40, 60, 80 and 100). The distribution of the fold-changes between the predictions and observed reality in each of the five bins was computed using a gaussian kernel density estimator available at Scikit Learn (bandwidth=0.1, remaining parameters with default values). The area under the obtained probability density function is equal to 1. Trendiness is the area of the right tail of the probability density function bounded to the left by the observed fold change. This provides an estimate of how extreme the fold change was for that gene in a specific bin.

Time-series data from 1980 to 2013 was used to predict the per gene publication dynamics in each category between 2014 and 2019 using a Recurrent Neural Network model with an encoder-decoder architecture preceded by an attention layer, where both the encoder and decoder are composed of five hidden layers of Gated Recurrent Units (GRU). The model was implemented in Keras using the Tensorflow-GPU backend. Min-max normalization was used to rescale the time series before training. The optimizer was RMSprop and the loss was computed as the log error. 30% of the time series was reserved for validation during the training.

Input data was in both forms: cumulative and differential. Multiple normalizations were used (‘none’, ‘minmax’, ‘log’, ‘standard’, and combinations of them). Similar results were obtained with different normalization and minmax was finally selected. Multiple Recurrent Neural Networks (RNNs) architectures were used (GRU, LSTM) in the form of encoder-decoder, with different numbers of neurons (1, 5, 10, 20, 50). Models were compared with the Mean Accuracy Scaled Error (MASE), an unbiased method to compare time-series prediction models by comparing how much each model outperforms a naive model that repeats the last value. The 5-neuron-GRU was selected because it was the most parsimonious model with the smallest MASE.

To identify trendy genes of pharmaceutical interest, the normalized mutual information of genes and diseases in the titles and abstracts of publications were computed. Many trendy genes cluster forming trendy pathways when obtaining gene-gene and gene-disease association networks. Enrichment of gene ontology (GO) terms for biological processes is used to uncover common pathways among the top 100 trendiest genes. Among the most enriched GO terms in both academia and pharma are T cell costimulation, execution phase of necroptosis, and pyroptosis. These biological processes are enriched in trendy genes, which may reflect that these fields of study are generating the most innovation and expectations in current biomedical research.

After the detection of gene trends, the next step was to understand why those genes might be trendy and curate possible mistakes in the disambiguation. With this aim, a topic detection pipeline was implemented as an automatic, fast discovery tool to study groups of publications that mention the gene of interest. In this context, topic modelling algorithms were used. A topic is a collection of similar words, specific to a group of documents. Two different topic detection algorithms were used: Latent Dirichlet Allocation (LDA), and Non-Negative Matrix Factorization (NMF). Both algorithms factor a nonnegative matrix ‘A’ with size N×M, where N is the number of publications and M is the dimension of the TF IDF vector obtained for Named Entity Recognition, into non-negative factors matrix W of size N×K and matrix H with size K×M, where W×H is an approximation of matrix A. The matrix W contains the strength of the association of a given publication to belong to a latent topic while H contains the strength of the association between a latent topic and a given n-gram. Scikit Learn implementations for both algorithms were used to generate ‘K’ number of topics defined by the user with the default parameters until convergence (tolerance of 1e-12). Topic timelines were obtained by calculating the mean and standard deviations of the topic probabilities for all publications mentioning the gene of interest per calendar year.

A review recommender system to accelerate the screening of the publications that cover most of the information in a network can also be designed. There are an average of 2.9 reviews citing any publication that mentions at least one gene name. The aim was to minimize the time reading and maximizing the information within a gene subnetwork. The algorithm aggregates both topic and network information from the citation subgraph of the publications that mention the gene of interest to obtain the most query-centric reviews. The topic information comes from the latent topics obtained from the topic detection algorithm. The topic probability of the publications and an aggregated PageRank score of the citation networks was used. The network information was captured by the PageRank scores of the subgraph. The user can select an interval number of reviews (R) that they are willing to read: between 2-3 or 3-50. Then, three matrices are defined for each group of publications: (i) a binary, sparse matrix of size N×R with N publications and R reviews that comprised the citation adjacency network; (ii) a N×1 weight matrix that comprise a PageRank scores; and (iii) a N×K matrix with the topic probabilities for N publications and K user-defined topics. The score for each review was defined as the sum of the PageRank scores of its references while the score for a combination of reviews is defined as the row sum of the indexed N×R matrix multiplied by the N×1 PageRank vector and the sum of the obtained vector. Results were later normalized by the total maximum score, defined as a hypothetical review citing all gene publications. For this method, the best reviews are the ones that cite the publications with highest PageRank scores. Finally, to minimize the number of reviews the combination that simultaneously maximizes the cumulative PageRank score and minimizes the overlapping of their combined citations was found. This way, a small set of reviews covering the main topics and publications in the field can be obtained. This recommender system can be used to select the optimal subset of reviews to assess why genes might be trendy.

The number of publications per gene in aggregate is generally very predictable. However, occasionally genes present significantly more publications than expected, meaning that a recent breakthrough occurred which cannot be accounted for from the publication dynamics. A ‘trendiness’ metric can identify emerging targets from the literature for rapid profiling at genome-scale. The trendiness is combined with gene-disease associations to prioritize potential drug targets: emergent genes associated with diseases but yet included in pharmaceutical publications are worthy of being investigated as potential targets. It is observed that trendy genes usually cluster into the same biological pathways.

In summary, the described example method includes downloading publication data from PubMed® baseline and creating a graph database with the acquired information. A comprehensive collection of human coding gene names and synonyms is acquired, and the method involves automatic determination of potential ambiguous (unsafe) gene names. The graph database is annotated with unambiguous gene symbols by combining co-citation network topology and binary classifiers. The method then involves prediction of per-gene publication trends using a recurrent neural network. When a gene has significantly more publications or citations than expected by the model it is considered to be ‘trendy’. The method optionally involves automatic topic detection of collections of publications, and this algorithm was used to quantify the evolution of topics in trendy gene publications over time. Optionally, a review recommender system that uses information from the citation network and topic detection to recommend the most efficient set of reviews to explore the literature can be implemented.

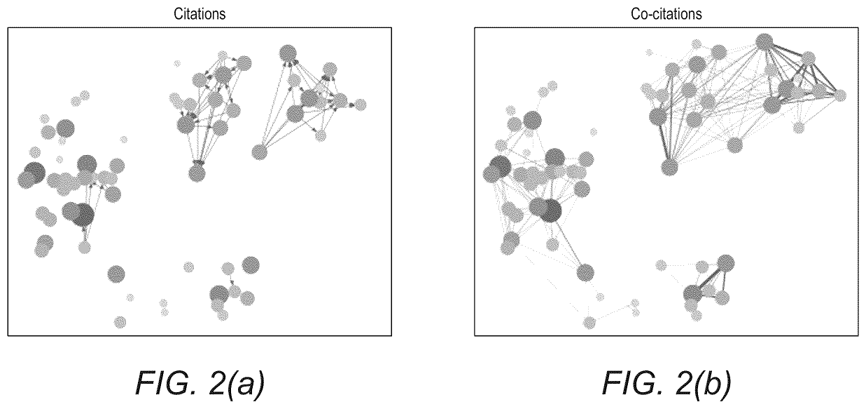

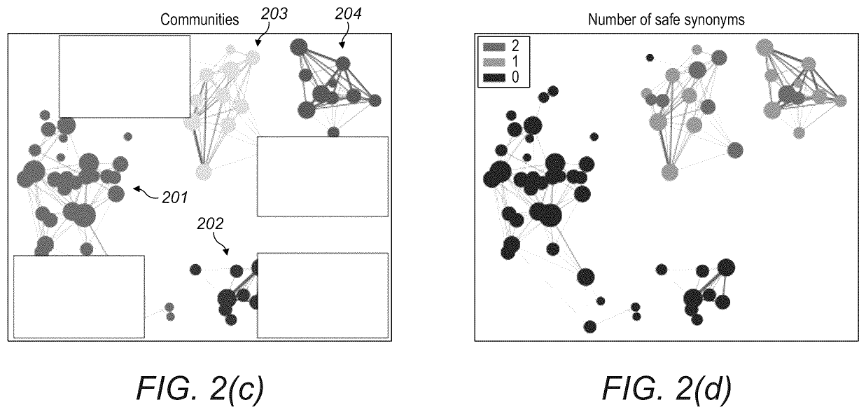

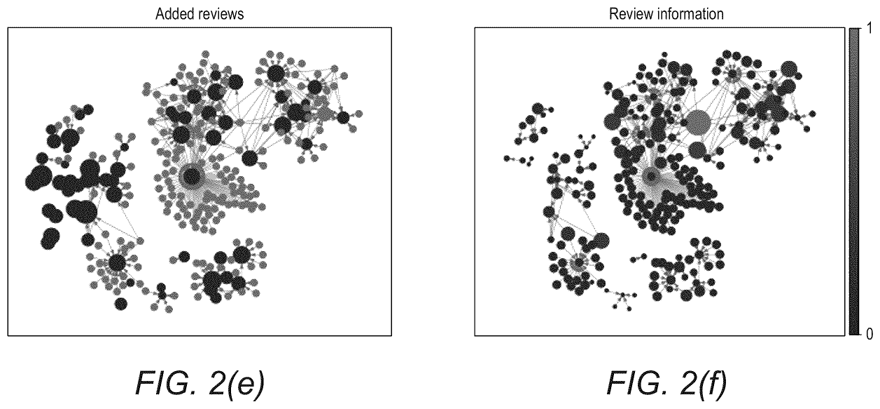

FIGS. 2(a)-2(f) illustrate an example of the created graph database for a particular gene when different ones of the above-described techniques or steps are used. In particular, FIG. 2(a) illustrates a citation network for a subset of publication documents from PubMed® mentioning any of the gene synonyms of the gene symbol LRWD1, including ORCA. The nodes represent publication documents and the size of the nodes represent the number of citations. The edges indicate citations between documents, including the direction of the citation. FIG. 2(b) illustrates a co-citation network of the same subset of publication documents as in FIG. 2(a). The thickness of the edges represents the number of times a pair of documents have been co-cited. FIG. 2(c) illustrates different communities of publication documents obtained by using iGraph's fast greedy algorithm, as described above. Each community is associated with different obtained topics. For instance, there is a so-called ‘killer whale’ community 201, an ‘orca plant’ cluster or community 202, and ‘LRWD1 in drosophila’ community 203, and an ‘LRWD1 in heterochromatin’ community 204. FIG. 2(d) indicates the number of safe synonyms in the title or abstract of each publication document in the same co-citation network. FIG. 2(e) illustrates the citation network with review documents added to show citations by the review documents to any of the publication documents. FIG. 2(f) illustrates review information as defined by the recommender system scaled from 0 to 1.

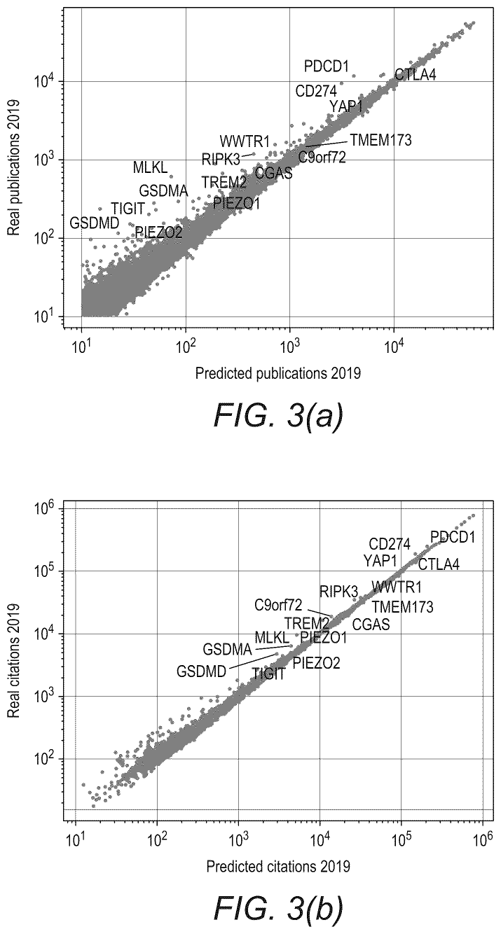

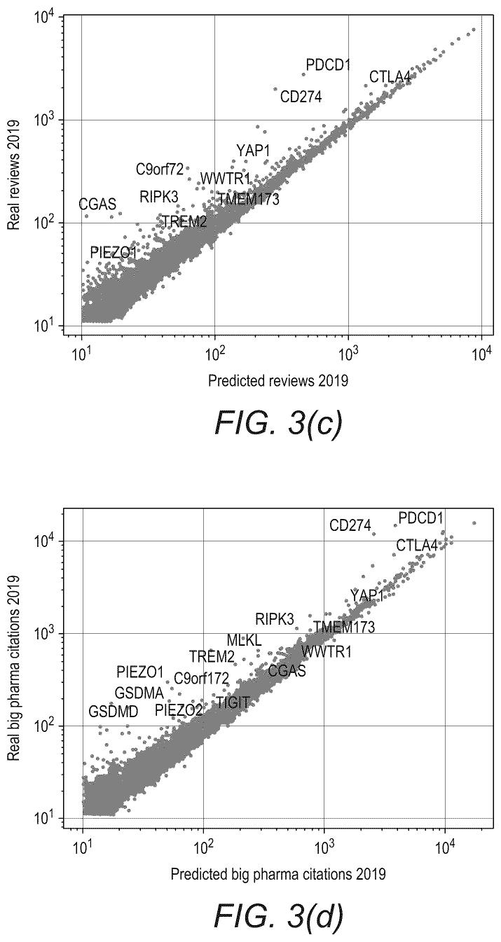

FIGS. 3(a)-3(d) illustrate the detection of trends of different genes, and of gene-gene-disease co-occurrence. In particular, FIG. 3(a) shows a logarithmic scatter plot of the predicted number of publications against the real number of publications in the year 2019 for different genes. Similarly, FIGS. 3(b), 3(c), and 3(d) respectively show the predicted number of review documents, citations, and citations from ‘big’ pharmaceutical companies against the actual number of review documents, citations, and citations from ‘big’ pharmaceutical companies in the year 2019 for different genes. Those genes whose real number of publications, for instance, is greater than the predicted value (i.e., whose node is above a line indicating a log linear relationship) may be considered trendy and of interest as potential drug targets.

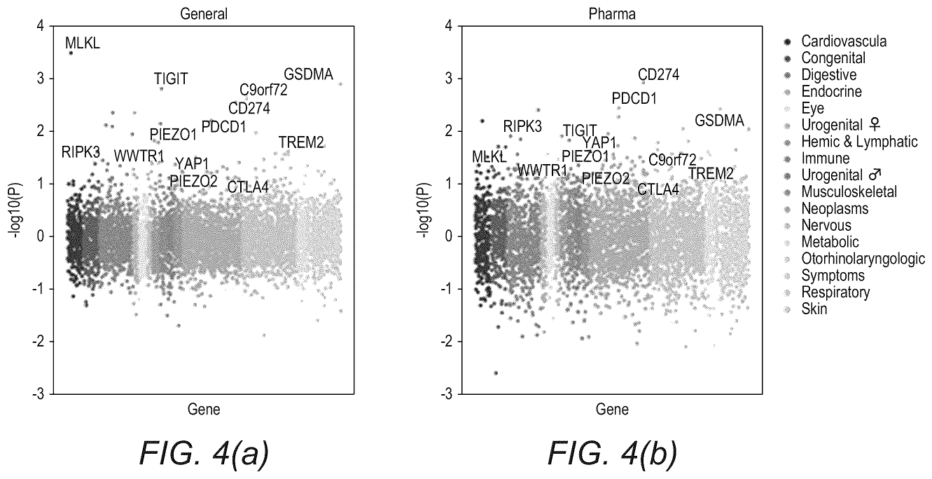

FIGS. 4(a) and 4(b) illustrate the trendiness—namely, log₂ (predicted/real)—for different genes associated with different groups of diseases (according to MeSH parent categories). In particular, FIG. 4(a) illustrates average trendiness of publications, reviews, citations, and citations from reviews for all (general) publication documents, and FIG. 4(b) illustrates average trendiness of publications, reviews, citations, and citations from reviews originating from big and medium sized pharmaceutical companies.

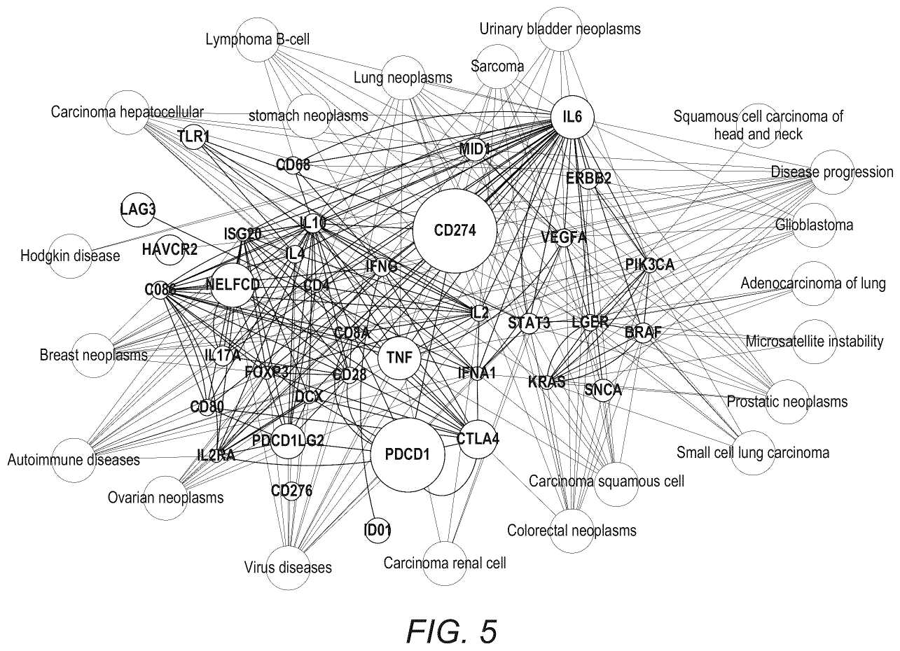

FIG. 5 illustrates a gene-gene-disease co-occurrence network of the first neighbors of CD274. Disease and gene nodes are labelled with their defined name, and the size of the gene nodes represents their ‘trendiness’ according to the define metric. The edges indicate gene-disease and gene-gene associations, with the width of the edges reflecting the number of co-occurrences in each case.

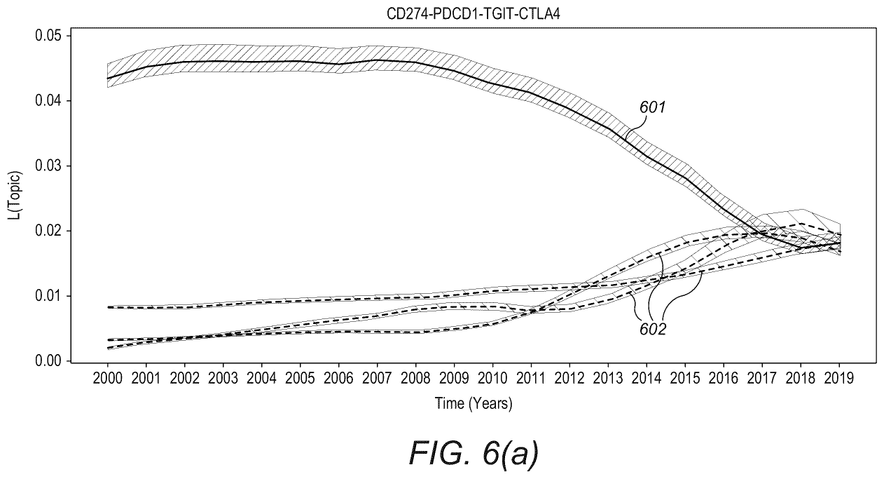

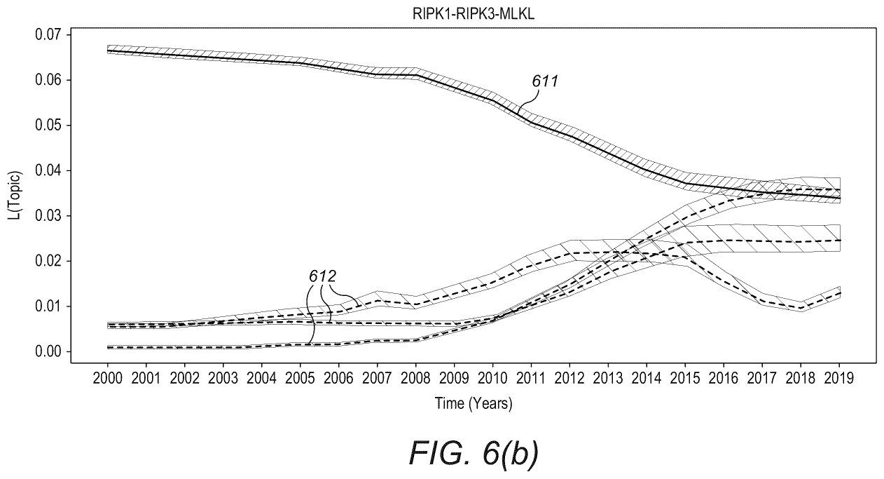

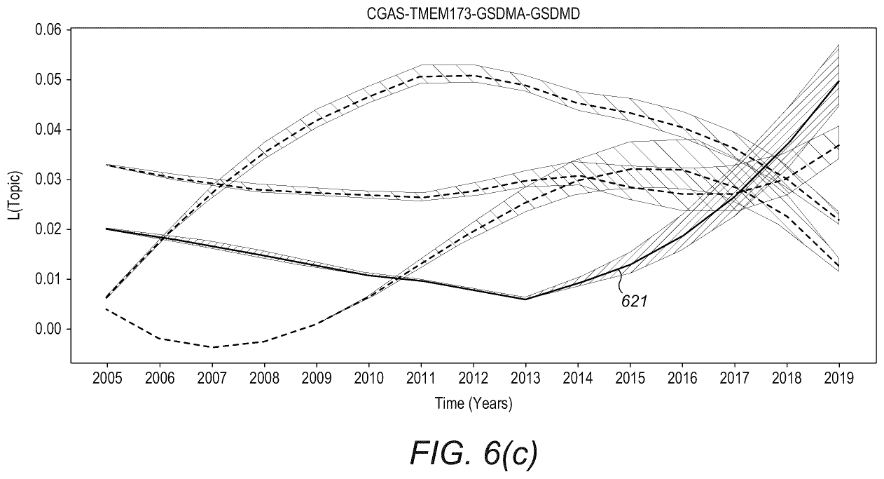

FIGS. 6(a)-6(c) illustrate topic timelines for the number of publications mentioning different genes of interest, i.e., the evolution of the topics associated with some trendiest genes are explored. In particular, FIGS. 6(a), 6(b), and 6(c) respectively show topic timelines for publications mentioning any of the genes for the immune checkpoint inhibitor, necroptosis, and pyroptosis pathways. In each case, four topic timelines are shown. The latent four topics were obtained using Non-Negative-Factorization of all publications annotated with the genes after disambiguation. All timelines show a rising topic after 2013 that represents the reason why these genes became ‘trendy’.

With reference to FIG. 6(a), for the immune checkpoint inhibitors (CD274, PDCD1, TGIT and CTLA4) the topic timeline suggests that there was a rapid decrease in the likelihood of publications—indicated by the topic timeline labelled 601—discussing the biological role of these immune checkpoint inhibitors since 2010, which coincides with a notable increase in topics—labelled 602—that discuss cancer therapies and monoclonal antibodies that target these four different transmembrane immunoglobulins. In this way, the topic-detection pipeline is able to capture the evolution of the research from its biological description to the clinical application.

With reference to FIG. 6(b), the topic timeline of the members of the necroptosis pathway (RIPK1, RIPK3 and MLKL) suggests that in the last decade there has been a decrease in the likelihood of publications discussing these genes in the context of apoptosis—indicated by the topic timeline labelled 611—in favour of publications that verse on the newly discovered form of cell death, the necroptotic pathway, as well as, the translational medicine perspective of this pathway as is suggested by words like mouse, treatment and activity or cancer (indicated by the topic timelines labelled 612).

With reference to FIG. 6(c), the topic timeline of the members of the pyroptosis pathway (CGAS, TMEM173, GSDMA and GSDMAD) shows a fast increase from 2013 of publications discussing the therapeutic opportunity in cancer immunotherapy with agonists for TMEM173 (indicated by the topic timeline labelled 621), while again, the remaining topics seemed to contain information on the biochemistry and biological role of the genes.

Some example case studies illustrating the above-described example method are described below.

Immune Checkpoint Inhibitors: CTLA4, CD274, PDCD1, TIGIT

CTLA4, PDCD1 (PD-1), CD274 (PD-L1) and TIGIT are among the trendiest genes in academia and pharma in 2019. CTLA4, PDCD1, CD274 and TIGIT genes encode four different transmembrane immunoglobulins that act as co-inhibitory receptors: checkpoints or ‘breaks’ for the adaptive immune response that prevent T cells from exerting their functions. CTLA4 competes with its analogous CD28 for CD80 and CD86 to prevent a premature activation of T cells. PDCD1-CD274 interaction counters the positive signals that may have already activated T effector cells. TIGIT interacts with CD155 to down-regulate natural killer cells and T lymphocytes. Cancer cells attempt to impair these checkpoints and currently there are 7 FDA approved monoclonal antibodies that target three of proteins (CTLA4: Ipilimumab; PDCD1: Nivolumab, Pembrolizumab, Cemiplimab; CD274: Atezolizumab, Avelumab) and multiple candidates targeting TIGIT (BGB-A1217, OMP-313M32, MTIG7192A, AB154).

Neurodegeneration: TREM2 and C9orf72

Recent discoveries are revolutionizing understanding of neurodegenerative diseases. C9orf72 encodes a guanine nucleotide exchange factor involved in endosomal trafficking and autophagy. Hexanucleotide repeat expansions in promoter or intronic regions of C9orf72 are some of the major causes of sporadic and familial forms of both amyotrophic lateral sclerosis and frontotemporal dementia. Antisense oligonucleotides are being used to impede the transcription of C9orf72 or CRISPR-Cas9 system to target the GGGGCC repeat in the DNA or RNA.

TREM2 gene encodes a transmembrane immunoglobulin receptor expressed in macrophages, osteoclasts, dendritic cells, and brain microglia. TREM2 variants have been associated with Nasu-Hakola disease, late-onset Alzheimer's disease, frontotemporal dementia, amyotrophic lateral sclerosis, and Parkinson's disease. TREM2 activates a pathway—through TYROBP/DAP12—that promotes inflammation and promotes phagocytosis of cellular waste, remains of apoptotic cells, and pathogens. Currently, two independent groups have generated anti-TREM2 antibodies to stimulate microglia to remove amyloid plaques. Furthermore, the mAb generated by one of these groups, Alenco, in collaboration with Abbvie, has entered Phase I clinical trials.

DNA Sensing by cGAS-STING: cGAS, TMEM173, GSDMD, GSDMA

The cytosolic nucleic acid-sensing pathway leads to pyroptosis, a lytic pro-inflammatory type of cell death involved in antiviral, antibacterial, and anticancer response. cGAS is a nucleotidyl-transferase that catalyses production of cyclic GMP-AMP (cGAMP) upon the recognition of double-stranded DNA. TMEM173 (STING) binds to cGAMP and promotes the activation of both TBK1 and IRF3, increasing the transcription of genes encoding type I interferons. GSDMA and GSDMD are pore-forming effector proteins in the plasma membrane to release proinflammatory interleukins like IL-1β and IL-18. The cGAS-STING pathway has been associated to multiple autoimmune and chronic inflammatory diseases like non-alcoholic fatty liver disease, systemic lupus erythematosus, vascular and pulmonary syndrome, macular degeneration, Bloom syndrome, Aicardi-Goutières syndrome, cancer, DNA damage, neurodegeneration and beyond. Currently, there are ongoing clinical trials for TMEM173 and GSDMD although there are no reported trials for GSDMA nor cGAS.

Necroptosis: RIPK1, RIPK3, and MLKL

RIPK1, RIPK3 and MLKL form part of the tumour necrosis factor-induced necroptosis pathway. This pathway has been associated with multiple pathologies: systemic inflammatory response syndrome, ulcerative colitis, psoriasis, rheumatoid arthritis, neurodegenerative diseases and even cancer. TNFR1, FasL, TRAIL, and TLR can all activate RIPK1 to decide the cell's fate: inflammation, apoptosis or necrosis. If caspase-8 is inhibited, RIPK1 and RIPK3 form the necrosome that subsequently phosphorylates MLKL. MLKL forms homo-trimers, migrates to the plasma membrane, binds to highly phosphorylated inositol phosphates, creates pores in the membrane and disrupts the cell integrity. The discovery of RIPK1 dates back to 1995. Since then, four inhibitor programs have progressed through human phase II safety trials. The first publication mentioning MLKL is more recent and, despite the lack of kinase activity, pharmaceutical companies have cited its publications by 60 times more since 2013. Although there are no clinical trials yet, there are at least three known different chemical inhibitors.

Mechanobiology: YAP1/WWTR1, PIEZO1 and PIEZO2

Cells use mechanical cues from their environment to guide behaviours such as proliferation and migration. Forces act as signals which are transduced to the nucleus where they control gene expression. Mechanical forces are critical regulators of organ and tissue homeostasis, morphogenesis, and regeneration, and are important aspects of diseases like cancer, metastasis, fibrosis, and cardiac hypertrophy. YAP1/WWTR1 (TAZ) are transcriptional co-activators and mechanotransducers. YAP/TAZ is hyperactivated in cancers, its inhibition reduces atherogenesis and fibrosis, it triggers pulmonary hypertension, and it is necessary for epithelial regeneration in the intestine. PIEZO1 and PIEZO2 are two mechano-sensitive cation channels that play a key role in cell number regulation and migration, hearing, neural and vascular development, somatosensory functions, proprioception and beyond. Piezo channels have been recently associated with multiple pathologies like arthrogryposis, apnea, congenital lymphatic dysplasia, hyperalgesia, malaria, pancreatitis, xerocytosis, Gordon syndrome, Marden-Walker Syndrome, and Distal Arthrogryposis Type 5. The discovery of mechanotransduction signaling pathways has received notable attention in the last years and may open the door to new therapeutic strategies to treat these diseases.

What is claimed is:

1. A method for computational drug target selection, comprising:
ingesting publication data, from at least one publication data source, relating to a plurality of publication documents, including historical publication documents and current publication documents;
searching the publication data to provide an indication, for each of the publication documents, as to whether the respective publication document is associated with one or more drug targets;
determining an expected publication parameter for each of the one or more drug targets based on the searched publication data from the historical publication documents, and determining an actual publication parameter for each of the one or more drug targets based on the searched publication data from the current publication documents; and
evaluating each of the one or more drug targets for selection based on its actual publication parameter relative to its expected publication parameter.

2. The method according to claim 1, further comprising:
defining, for each of the one or more drug targets, one or more character expressions referring to the respective drug target, wherein searching the publication data comprises searching the publication data for the one or more character expressions for each of the one or more drug targets.

3. The method according to claim 2, further comprising:
for each of the one or more drug targets:
classifying each of the one or more character expressions corresponding to the respective drug target as a safe character expression or an unsafe character expression, wherein the classification is based on a likelihood that an instance of a respective character expression in the publication data refers to the respective drug target, and wherein, if the searched publication data from one of the publication documents includes a safe character expression, then the publication document is determined to be associated with the drug target.

4. The method according to claim 2, wherein one or more character expression unsafe characteristics are user-defined to indicate that a corresponding character expression is unsafe, and wherein character expressions in the searched publication data that exhibit one or more of the character expression unsafe characteristics are classified as unsafe character expressions.

5. The method according to claim 2, wherein one or more character expression ambiguity characteristics are defined to ascribe an ambiguity score to one or more of the character expressions, and wherein each of the character expressions is classified as a safe character expression or an unsafe character expression based on the corresponding ascribed ambiguity score.

6. The method according to claim 5, further comprising:
applying a machine learning algorithm to ascribe the ambiguity score to each of the one or more character expressions based on one or more character expression ambiguity characteristics, wherein the machine learning algorithm uses the one or more character expression unsafe characteristics to ascribe the ambiguity score to each of the one or more of the character expressions, and wherein the machine learning algorithm comprises a positive-unlabeled learning technique.

7. The method according to claim 1, wherein:
the publication data for at least some of the publication documents includes citation data indicative of citations made by one publication document to one or more other publication documents from the plurality of publication documents; and
searching the publication data comprises identifying, using the citation data, pairs of publication documents that have been cited by the same publication document.

8. The method according to claim 7, further comprising:
determining, for each identified pair of publication documents, a co-citation value representative of a number of publication documents that cite both of the publication documents of the respective identified pair of publication documents.

9. The method according to claim 8, further comprising:
assigning pairs of publication documents to one of a plurality of communities of publication documents based on their determined co-citation value and on the publication documents that cite the pairs of publication documents.

10. The method according to claim 9, further comprising:
defining, for each of the one or more drug targets, one or more character expressions referring to the respective drug target, wherein searching the publication data comprises searching the publication data for the one or more character expressions for each of the one or more drug targets; and
determining, for each of the plurality of communities of publication documents, whether to associate the community with one of the drug targets, wherein the determination comprises determining which of the defined character expressions referring to the one drug target are present in the publication data of each of the publication documents in the community.

11. The method according to claim 10, further comprising:
for each of the one or more drug targets:
classifying each of the one or more character expressions as a safe character expression or an unsafe character expression, wherein the classification is based on a likelihood that an instance of the character expression in the publication data refers to the drug target, and wherein determining whether to associate the community with one of the drug targets comprises determining a proportion of the publication documents in the community that include at least one safe character expression in their publication data.

12. The method according to claim 7, further comprising:
defining, for each of the one or more drug targets, one or more character expressions referring to the respective drug target, wherein searching the publication data comprises searching the publication data for the one or more character expressions for each of the one or more drug targets, and wherein searching for the pairs of publication documents includes searching for pairs of publication documents that each includes at least one of the character expressions defined as referring to one of the drug targets.

13. The method according to claim 1, wherein determining the expected publication parameter comprises using a machine learning algorithm trained using the searched publication data from the historical publication documents.

14. The method according to claim 1, further comprising:
determining a target-target co-occurrence parameter between pairs of the drug targets, the target-target co-occurrence parameter being determined based on the indication from the searched publication data of which publication documents both drug targets in a pair are associated with, each target-target co-occurrence parameter being indicative of the number of publication documents in which both of the drug targets in a respective pair appear; and
evaluating the one or more drug targets for selection based on the determined target-target co-occurrence parameters.

15. The method according to claim 1, further comprising:
searching the publication data to provide an indication, for each of the publication documents, as to whether the respective publication document is associated with one or more diseases; and
determining a target-disease co-occurrence parameter between each of the drug targets and each of the diseases, the target-disease co-occurrence parameter being determined based on the indication from the searched publication data of which publication documents each drug target and each disease are associated with, each target-disease co-occurrence parameter being indicative of the number of publication documents in which one of the drug targets and one of the diseases appear; and
evaluating the one or more drug targets for selection based on the determined target-disease co-occurrence parameters.

16. The method according to claim 1, further comprising:
applying a topic modeling algorithm to the publication data for the publication documents associated with each of the drug targets to obtain one or more topics associated with each drug target; and
evaluating the one or more drug targets for selection based on the obtained one or more topics.

17. The method according to claim 1, wherein the publication data includes a publication date for each of the plurality of publication documents, and wherein the publication date defines whether each of the publication documents is a historical publication document or a current publication document.

18. The method according to claim 1, further comprising:
using the evaluation of the one or more drug targets to inform selection of at least one of the drug targets for use in a drug discovery project; and
designing the drug discovery project by selecting at least one of the drug targets for use in the drug discovery project based on the evaluation.

19. The method according to claim 18, further comprising:
undertaking the drug discovery project using the at least one selected drug target, wherein undertaking the drug discovery project includes selecting and testing compounds against the at least one selected drug target.

20. A computer device for drug target selection, the computer device configured to:
ingest publication data, from at least one publication data source, relating to a plurality of publication documents, including historical publication documents and current publication documents;
search the publication data to provide an indication, for each of the publication documents, as to whether the respective publication document is associated with one or more drug targets;
determine an expected publication parameter for each of the one or more drug targets based on the searched publication data from the historical publication documents, and determine an actual publication parameter for each of the one or more drug targets based on the searched publication data from the current publication documents; and
evaluate each of the one or more drug targets for selection based on its actual publication parameter relative to its expected publication parameter.
